(19) United States
(12) Patent Application Publication
(10) Pub. No.: US 2009/0187238 A1
(43) Pub. Date: Jul. 23, 2009

(54) SYSTEM AND METHOD FOR DEPLOYING SELF-EXPANDABLE MEDICAL DEVICE WITH COATING

(75) Inventors: Jan Weber, Maastricht (NL); Dominique Seidel, Munchen (DE)

Correspondence Address: KENYON & KENYON LLP, 1500 K STREET N.W., SUITE 700, WASHINGTON, DC, 20005, US

(73) Assignee: BOSTON SCIENTIFIC SCIMED, INC., Maple Grove, MN (US)

(21) Appl. No.: 12/354,067

(22) Filed: Jan. 15, 2009

Related U.S. Application Data
(60) Provisional application No. 61/021,801, filed on Jan. 17, 2008.

(51) Int. Cl. A61F 2/84

(52) U.S. Cl. 623/1.12

(57) ABSTRACT

A system and method for providing a coating on a self-expandable medical device such as a stent are disclosed. The system comprises a coating applicator at the distal end of a sheath which delivers coating material onto the stent as the stent is deployed from the sheath. Thus, the stent may be loaded into the sheath without a coating on the stent, thereby avoiding shearing off or damaging the coating during loading. Also, the coating is applied only as the stent exits the sheath, thereby avoiding shearing off or damaging the coating during deployment.

### CROSS REFERENCE TO RELATED APPLICATION

The present application claims priority to U.S. provisional application Ser. No. 61/021,801 filed Jan. 17, 2008, the disclosure of which is incorporated herein by reference in its entirety.

### FIELD OF INVENTION

The present invention provides methods and materials for providing a coating on a medical device.

### BACKGROUND

Medical devices may be coated so that the surfaces of such devices have desired properties or effects. For example, it may be useful to coat medical devices to provide for the localized delivery of therapeutic agents to target locations within the body, such as to treat localized disease (e.g., heart disease) or occluded body lumens. Localized drug delivery may avoid some of the problems of systemic drug administration, which may be accompanied by unwanted effects on parts of the body which are not to be treated. Additionally, treatment of the afflicted part of the body may require a high concentration of therapeutic agent that may not be achievable by systemic administration. Localized drug delivery may be achieved, for example, by coating balloon catheters, stents and the like with the therapeutic agent to be locally delivered. The coating on medical devices may provide for controlled release, which may include long-term or sustained release, of a bioactive material.

Aside from facilitating localized drug delivery, medical devices may be coated with materials to provide beneficial surface properties. For example, medical devices are often coated with radiopaque materials to allow for fluoroscopic visualization while placed in the body. It is also useful to coat certain devices to achieve enhanced biocompatibility and to improve surface properties such as lubriciousness.

Coatings have been applied to medical devices by processes such as dipping, spraying, vapor deposition, plasma polymerization, spin-coating and electrodeposition. Although these processes have been used to produce satisfactory coatings, they may in some cases have potential drawbacks. For example, it may be difficult to achieve coatings of uniform thicknesses, both on individual parts and on batches of parts.

A conventional self-expandable (SE) stent has an expanded form when not constrained. To deliver the stent to the desired location, the stent is compressed radially and loaded into a delivery system. Typically, an outer tubular sheath retains the compressed stent. The delivery system is tracked to the region of a vessel being stented. The stent is then released from its compressed state, by retracting the sheath and/or pushing the stent out of the sheath. When released from the constraint of the sheath, the stent self-expands back to its expanded form to scaffold the vessel wall.

### BRIEF DESCRIPTION

The present invention provides a system and method for applying a coating to a self-expandable medical device such as a stent.

During loading and deployment of self-expandable stents, there may be significant friction between the stent surface and the sheath. Because the self-expandable stent has a tendency to want to expand to its relaxed state, the stent presses outward against the inner surface of the sheath. Thus, when the stent is being loaded into or deployed out of the sheath, the friction forces may be significant. Longer stents may have higher frictional forces. In the case of coated self-expandable stents, these forces may be damaging to the coating. The coating on the self-expandable stent that is in contact with the inner surface of the sheath is subject to high shear forces during both loading and deployment. Therapeutic agent coatings for medical devices may be relatively soft, for example consisting of a mixture of biodegradable or stable polymers and drugs, or solely drugs. This soft coating can be stripped or damaged by contact with the sheath during loading or deployment.

The present invention provides a system and method for providing a coating on a self-expandable medical device such as a stent while avoiding the issues relating to coating damage from the sheath during loading and deployment. In an embodiment, the present invention comprises a coating applicator at the distal end of the sheath which delivers coating material onto a stent as the stent is deployed from the sheath. Thus, the stent may be loaded into the sheath without a coating on the stent, thereby avoiding shearing off or damaging the coating during loading. Also, the coating is applied only as the stent exits the sheath, thereby avoiding shearing off or damaging the coating during deployment.

### BRIEF DESCRIPTION OF THE DRAWINGS

FIGS. 1a and 1b show a delivery sheath with a distal coating applicator prior to deployment of the stent (FIG. 1a) and during deployment of the stent (FIG. 1b).

FIG. 2a shows a first embodiment of a coating applicator. FIG. 2b shows a cross-sectional view of the device of FIG. 2a taken along the line A-A. FIG. 2c shows the stent after placement within a body curvature.

FIG. 3a shows a second embodiment of a coating applicator. FIG. 3b shows a magnified view of the a coating applicator of FIG. 3a. FIG. 3c shows a cross-sectional view of the device of FIG. 3a taken along the line B-B.

FIG. 4 shows a third embodiment of a coating applicator.

FIG. 5 shows an embodiment with multiple coating applicators for applying a multi-layer coating.

### DETAILED DESCRIPTION

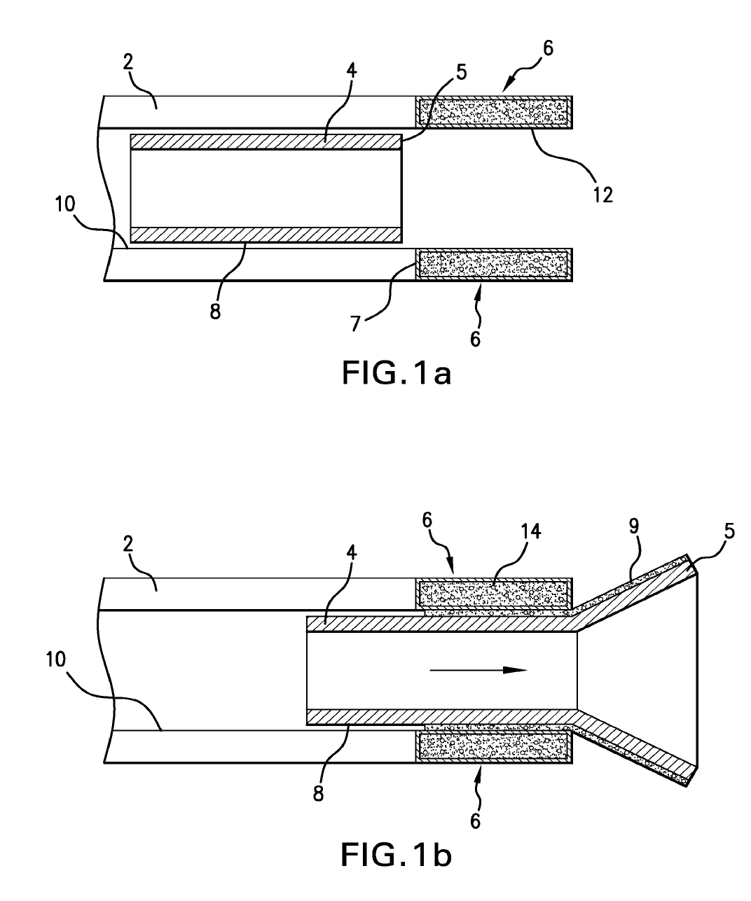

FIGS. 1a and 1b show an embodiment of a system for deploying a self-expandable stent with a coating. The system comprises a tubular sheath 2 and self-expandable stent 4. FIG. 1a shows a bare, un-coated, self-expandable stent 4 loaded into the sheath 2 before the sheath 2 is inserted into the body lumen. A coating applicator 6 in the form of a reservoir is mounted at the distal end 7 of the sheath 2.

For clarity, the stent 4 is shown schematically both in its structure and in its relation to the sheath. The stent may take any suitable configuration, and many such configurations are known in the art. It will be appreciated that the because a self-expandable stent 4 has a tendency to want to expand to its relaxed state, the outer surface 8 of the stent 4 ordinarily presses outward against the inner surface 10 of the sheath 2. For clarity of illustration, the figures show a small space between the outer surface 8 of the stent 4 and the inner surface 10 of the sheath 2, although it will be understood that in practice these surfaces will ordinarily be abutting.

As shown in FIG. 1b, when the stent 4 is deployed from the sheath 2 (by retracting the sheath 2 or pushing the stent 4 out of the sheath 2), the outer surface 8 of the stent 4 moves relative to the inner surface 10 of the sheath 2. Beginning at the distal end 5 of the stent 4, the stent 4 exits the sheath 5 through the opening at the distal end 7 of the sheath 2. The outer surface 8 of the stent 4 then contacts the annular inner surface 12 of the coating applicator 6, allowing the coating material 14 contained in the reservoir to be coated onto the outer surface 8 of the stent 4 during stent deployment. As the stent 4 exits the coating applicator 6, the distal end 5 of the stent 4 is allowed to expand toward its unconstrained shape to scaffold the body lumen.

It will be appreciated that the application of the coating material to the stent is illustrated schematically. The coating material may be applied gradually from the location where the stent enters the coating applicator to the location where the stent exits the coating applicator.

To facilitate the adhesion of the coating material to the outer surface of the stent, the outer surface of the stent may be porous or roughened. The coating applied by the applicator may comprise a therapeutic agent. As described in detail below, the coating applicator may take different forms.

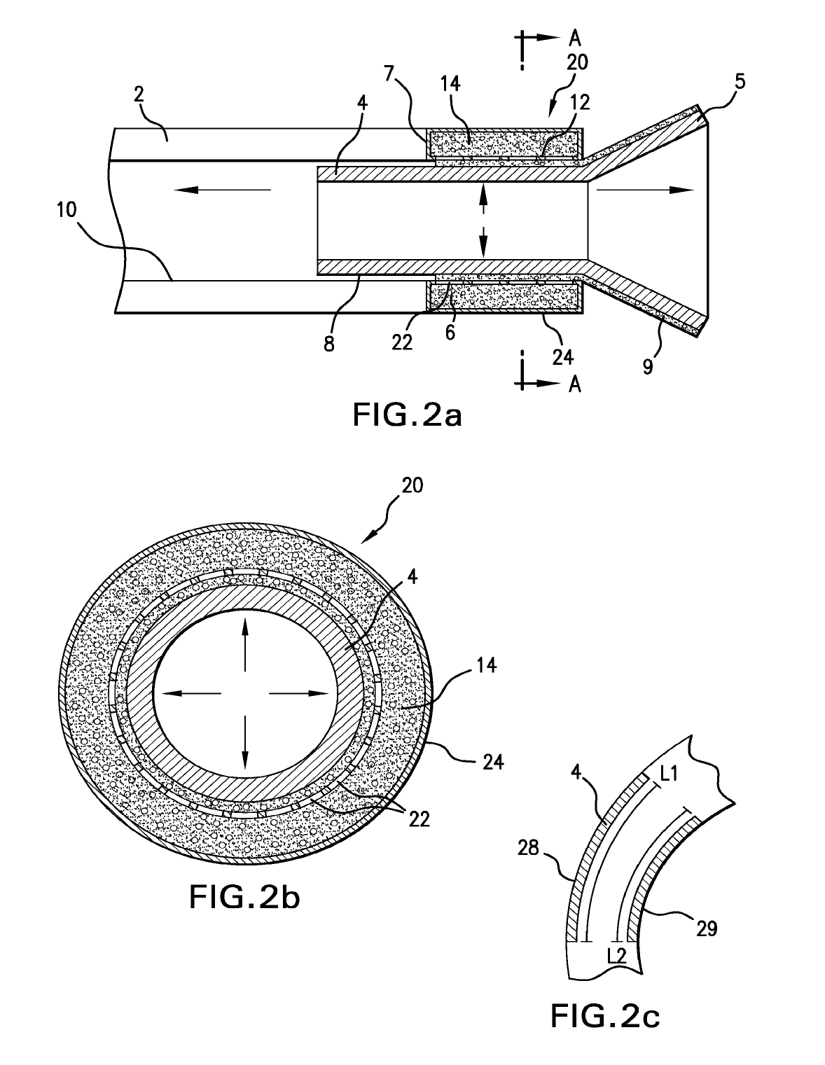

As shown in FIGS. 2a and 2b, one embodiment of a coating applicator 6 is a hollow annular tube or ring 20 with small holes 22 on the annular inner surface 12. Annular ring 20 has an outer surface 24 and an inner surface 12. The inner surface 12 may be relatively flexible and deformable, and the outer surface 24 may be relatively hard as compared to the inner surface 12. As the self-expandable stent 4 is deployed from the sheath 2 it exerts a radial pressure on the annular ring 20, causing the annular ring 20 to be slightly compressed. Compression of the annular ring 20 causes the coating material 14 to be expelled through holes 22 on the inner surface 12.

The holes 22 may be, for example, about 1 μm in diameter, but other sizes are of course possible. In addition, the holes may be arranged around the entire circumference of the inner surface 12 to deliver a uniform coating. Alternatively, the annular ring 20 can have holes 22 of a varying diameter and/or density to deliver more coating material 14 to a certain portion of the outer surface 8 of the stent 4. For instance, if the target site 26 within the body is known to have a curvature as shown in FIG. 2c, the stent portion along outer curvature 28 will have a length L1 that is greater than the length L2 of the stent portion along the inner curvature 29. Thus, the stent portion along the outer curvature 28 will be stretched more that the stent portion along the inner curvature 29. To achieve a relatively uniform dosing of therapeutic agent, the portion of the stent 4 that will be disposed on the outer curvature 28 may be coated with more therapeutic agent to achieve the same amount per unit area as the stent portion along the inner curvature 29. As another example, if a bifurcated stent is used, there may be a desire for more drug at the bifurcation of the body lumen. As yet another example, if the target site is known to have vulnerable plaque, more drug can be delivered to the portion of the stent 4 that will contact this plaque once the stent 4 is in place.

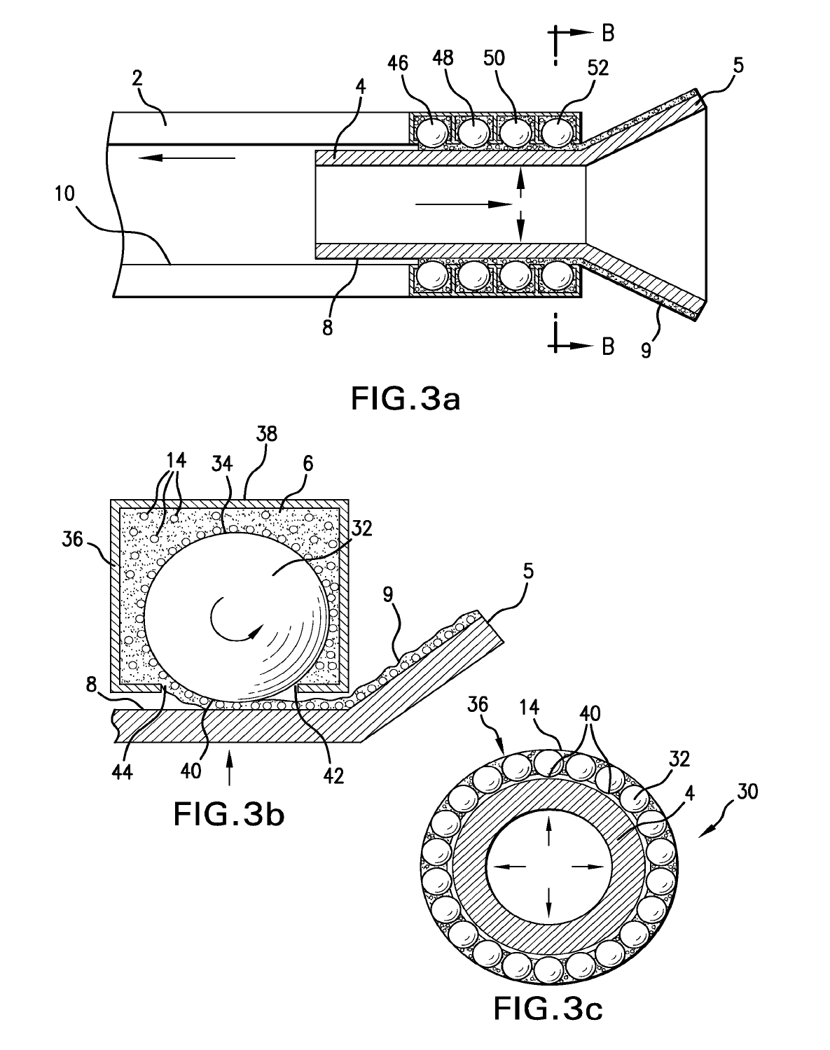

A second embodiment of a coating applicator 6 containing a coating material 14 comprises a one or more ball assemblies 30, shown in FIGS. 3a, 3b, and 3c. Ball assemblies 30 somewhat resemble ball bearings and operate similar to a ballpoint pen. Each ball assembly 30 comprises a plurality of spherical balls 32, each having an outer surface 34, with the spherical balls 32 arranged around the circumference of coating applicator 6. Spherical balls 32 can be mounted within a modified ball bearing housing 36 that holds them in place but allows them to rotate. The spherical balls 32 are caused to rotate under the forces caused by the longitudinal movement of the stent 4 as it is deployed from the sheath 2.

The housing 36 can contain the coating material 14 within it. Alternatively, the top 38 of the housing 36 can be open to receive the coating material 14 from a separate reservoir. Alternatively, the top 38 of the housing 36 can have a sponge-like material to assist applying the coating material to the spherical balls 32 as the balls 32 rotate.

The spherical balls 32 are housed almost entirely within the housing 36, but a coating portion 40 protrudes from the housing 36 and is designed to contact the outer surface 8 of the stent 4. The outer surface 34 of spherical balls 32 carries coating material 14 from the reservoir, and this coating material is transferred to the outer surface 8 of stent 4 upon contact. The coating material 14 will stick to the outer surface 8 of the stent 4. The transfer of coating material may be facilitated by having the stent outer surface roughened or porous; by comparison, the surface of the spherical balls 32 may be relatively smooth.

The coating portion 40 changes as the spherical balls 32 rotate, and is defined as the portion located between the proximal side 44 and the distal side 42 of the opening in the housing 36. Due to the relative movement between the spherical balls 32 and the stent 4, the clearance between the spherical balls 32 and the housing 36 at the proximal side 44 of the opening is greater than the clearance between the spherical balls 32 and the housing at the distal side 42 of the opening. Thus, when a portion of a spherical ball 32 rotates into the housing at the distal side 42 of the opening, the housing may shear coating material off of the spherical ball 32 to help force coating material to remain on the stent 4.

The coating applicator 6 can include more than one ring of balls 32 arranged sequentially, as shown in FIG. 3a. In one embodiment there are four rings of balls, 46, 48, 50 and 52. The stent 4 contacts the proximal-most ring 46 first, the central rings 48, 50 next, and the distal-most ring 52 last. Although this embodiment is shown with four rings, more of fewer rings could be used. The balls within the rings 46, 48, 50 and 52 may be staggered relative to each other to adequately coat the entire outer surface 8 of stent 4. Thus, for example, space between the balls 32 that may be left uncoated by the proximal-most ring 46 may be coated by the central ring 48; space that may be left uncoated by the central ring 48 may be coated by the central ring 50; and space that may be left uncoated by the central ring 50 may be coated by the distal-most ring 52.

The spherical balls 32 may have, for example, a diameter of about 150 μm, and the diameter of the ball assembly 30 may be, for example, about 2 mm. The balls can be made of any suitable material, which may be bio-compatible or coated with a bio-compatible material, and may be, for example, steel, carbon steel, chrome steel, stainless steel, cast iron steel, tungsten carbide, titanium, aluminum, hastelloy, cobalt, brass, phosphor bronze, glass, rubber, ceramic, zirconium. Other suitable dimensions and materials are of course possible. The balls 32 may be solid or hollow. Spherical balls of suitable size are known and available and may be obtained, for example, from DIT Holland B. V. (Hilvarenbeek, Netherlands) or JSK°Nanoball (Wermelskirchen, Germany).

In an alternative configuration, instead of using spherical balls 32, a number of cylindrical elements could be used. Alternatively, an O-ring made of elastic or other suitable material could be used in place of the spherical balls 32, wherein the O-ring extends around the circumference of the ring(s).

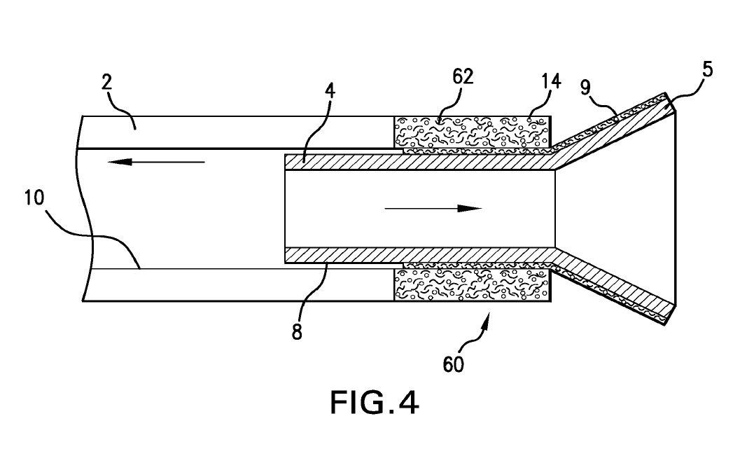

In a third embodiment of the coating applicator 6, the entire coating applicator 6 is formed from a delivery medium 60, which in this example is a gel 62. The gel 62 can be any biocompatible substance with a high viscosity that will not react with the therapeutic agent 14, and may be, for example, silicone gel or oil. The gel 62 may be embedded with therapeutic agent 14, as shown in FIG. 4. As the stent 4 contacts the delivery medium 60, a portion of the gel 62 including the therapeutic agent 14 will rub off onto the outer surface 8 of the stent 4 due to the shear forces exerted. As the stent 4 is deployed, the medium 60 will be slowly depleted as it forms a coating 9 on the stent 4. The delivery medium can have varying amounts of therapeutic agent 14 in different areas of the gel 62, as shown in FIG. 4, to deliver more therapeutic agent 14 to a certain portion of the outer surface 8 of the stent 4. Alternatively, the gel 62 can have the therapeutic agent 14 evenly distributed throughout. Alternatively, the gel 62 can have more than one therapeutic agent 14 dispersed throughout, with each therapeutic agent being in a distinct area or all the therapeutic agents being evenly distributed throughout. In alternative arrangements, the coating applicator 6 may be in the form of a sponge or made of sponge-like material, carrying therapeutic agent. In such an arrangement, the pressure of the stent on the coating applicator causes the therapeutic agent to be applied to the stent, similar to the operation of FIGS. 2a-2b or FIG. 4.

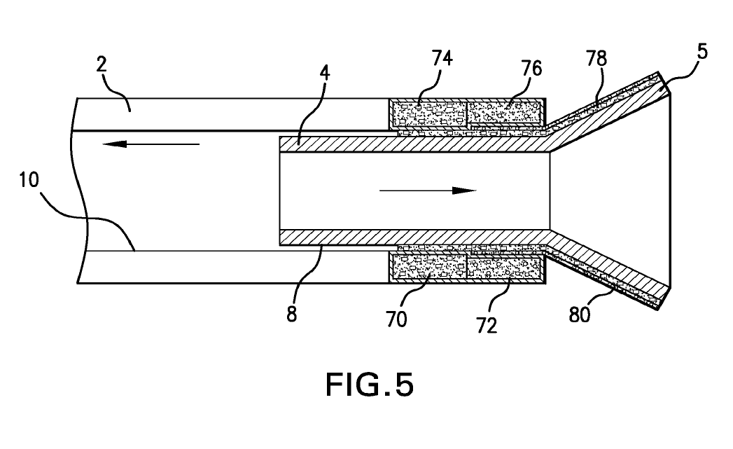

A further embodiment, shown in FIG. 5, includes more than one coating applicator in sequence to apply a multi-layer coating to the outer surface 8 of the stent 4 upon delivery. For example, a first coating applicator 70 and a second coating applicator 72 can be arranged sequentially, with first coating applicator 70 located proximal to second coating applicator 72. First coating applicator 70 may hold first coating material 74 and second coating applicator 72 may hold second coating material 76. In one embodiment, first coating material 74 comprises a slow-release drug forming an inner coating layer 78 and second coating material 76 comprises an immediate release drug forming an outer coating layer 80, which allows for an initial peak drug delivery and controlled drug delivery thereafter. In another embodiment, first coating material 74 comprises a mixture of drug and polymer forming the inner layer 78, and a second coating material 76 comprises a crosslinker forming the outer layer 80. In another embodiment, first coating material 74 comprises a anti-thrombogenic drug forming the inner layer 78, and a second coating material 76 comprises a anti-inflammatory drug forming the outer layer 80. Although two coating applicators are illustrated, any number of coating applicators could be arranged to provide multiple layers of coating to the stent 4.

A typical stent 4 may have a length, for example, of about 20-40 mm and a wall thickness of about 80-100 μm. The coating material 14 coating may be applied, for example, in an amount of 1 μg/mm². The coating applicator 6 can be mounted onto the distal end 7 of the sheath 2 by any conventional method, such as gluing, welding, mechanically fixing, melting the end of the sheath, or interference fit. The therapeutic agent in a coating of a medical device of the present invention may be any pharmaceutically acceptable agent such as a non-genetic or genetic therapeutic agent, a biomolecule, a small molecule, or cells.

Exemplary non-genetic therapeutic agents include anti-thrombogenic agents such as heparin, heparin derivatives, prostaglandin (including micellar prostaglandin E1), urokinase, and PPack (dextrophenylalanine proline arginine chloromethyl ketone); anti-proliferative agents such as enoxaparin, angiopeptin, sirolimus (rapamycin), tacrolimus, everolimus, zotarolimus, monoclonal antibodies capable of blocking smooth muscle cell proliferation, hirudin, and acetylsalicylic acid; anti-inflammatory agents such as dexamethasone, rosiglitazone, prednisolone, corticosterone, budesonide, estrogen, estradiol, sulfasalazine, acetylsalicylic acid, mycophenolic acid, and mesalamine; anti-neoplastic/anti-proliferative/anti-mitotic agents such as paclitaxel, epothilone, cladribine, 5-fluorouracil, methotrexate, doxorubicin, daunorubicin, cyclosporine, cisplatin, vinblastine, vincristine, epothilones, endostatin, trapidil, halofuginone, and angiostatin; anti-cancer agents such as antisense inhibitors of c-myc-oncogene; anti-microbial agents such as triclosan, cephalosporins, aminoglycosides, nitrofurantoin, silver ions, compounds, or salts; biofilm synthesis inhibitors such as non-steroidal anti-inflammatory agents and chelating agents such as ethylenediaminetetraacetic acid, O,O′-bis(2-aminoethyl) ethyleneglycol-N,N,N′,N′-tetraacetic acid and mixtures thereof; antibiotics such as gentamicin, rifampin, minocycline, and ciprofloxacin; antibodies including chimeric antibodies and antibody fragments; anesthetic agents such as lidocaine, bupivacaine, and ropivacaine; nitric oxide; nitric oxide (NO) donors such as linsidomine, molsidomine, L-arginine, NO-carbohydrate adducts, polymeric or oligomeric NO adducts; anti-coagulants such as D-Phe-Pro-Arg chloromethyl ketone, an RGD peptide-containing compound, heparin, anti-thrombin compounds including anti-thrombin antibodies, platelet receptor antagonists, anti-platelet receptor antibodies, enoxaparin, hirudin, warfarin sodium, dicumarol, aspirin, prostaglandin inhibitors, platelet aggregation inhibitors such as cilostazol and tick antiplatelet factors; vascular cell growth promoters such as growth factors, transcriptional activators, and translational promoters; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogenous vasoactive mechanisms; inhibitors of heat shock proteins such as geldanamycin; angiotensin converting enzyme (ACE) inhibitors; beta-blockers; PAR kinase (PARK) inhibitors; phospholamban inhibitors; protein-bound particle drugs such as ABRAXANE™; and any combinations and prodrugs of the above.

Exemplary biomolecules include peptides, polypeptides and proteins; oligonucleotides; nucleic acids such as double or single stranded DNA (including naked and cDNA), RNA, antisense nucleic acids such as antisense DNA and RNA, small interfering RNA (siRNA), and ribozymes; genes; carbohydrates; angiogenic factors including growth factors; cell cycle inhibitors; and anti-restenosis agents. Nucleic acids may be incorporated into delivery systems such as, for example, vectors (including viral vectors), plasmids or liposomes.

Non-limiting examples of proteins include SERCA 2 protein, monocyte chemoattractant proteins (“MCP-1”) and bone morphogenic proteins (“BMPs”), such as, for example, BMP-2, BMP-3, BMP-4, BMP-5, BMP-6 (VGR-1), BMP-7 (OP-1), BMP-8, BMP-9, BMP-10, BMP-11, BMP-12, BMP-13, BMP-14, BMP-15. Preferred BMPs are any of BMP-2, BMP-3, BMP-4, BMP-5, BMP-6, and BMP-7. These BMPs can be provided as homodimers, heterodimers, or combinations thereof, alone or together with other molecules. Alternatively, or in addition, molecules capable of inducing an upstream or downstream effect of a BMP can be provided. Such molecules include any of the “hedgehog” proteins, or the DNAs encoding them. Non-limiting examples of genes include survival genes that protect against cell death, such as anti-apoptotic Bcl-2 family factors and Akt kinase; serca 2 gene; and combinations thereof. Non-limiting examples of angiogenic factors include acidic and basic fibroblast growth factors, vascular endothelial growth factor, epidermal growth factor, transforming growth factors α and β, platelet-derived endothelial growth factor, platelet-derived growth factor, tumor necrosis factor α, hepatocyte growth factor, and insulin-like growth factor. A non-limiting example of a cell cycle inhibitor is a cathepsin D (CD) inhibitor. Non-limiting examples of anti-restenosis agents include p15, p16, p18, p19, p21, p27, p53, p57, Rb, nFkB and E2F decoys, thymidine kinase and combinations thereof and other agents useful for interfering with cell proliferation.

Exemplary small molecules include hormones, nucleotides, amino acids, sugars, and lipids and compounds that have a molecular weight of less than 100 kD.

Exemplary cells include stem cells, progenitor cells, endothelial cells, adult cardiomyocytes, and smooth muscle cells. Cells can be of human origin (autologous or allogeneic) or from an animal source (xenogeneic), or genetically engineered. Non-limiting examples of cells include side population (SP) cells, lineage negative (Lin⁻) cells including Lin⁻ CD34⁻, Lin⁻CD34⁺, Lin⁻c Kit⁺, mesenchymal stem cells including mesenchymal stem cells with 5-aza, cord blood cells, cardiac or other tissue derived stem cells, whole bone marrow, bone marrow mononuclear cells, endothelial progenitor cells, skeletal myoblasts or satellite cells, muscle derived cells, G₀ cells, endothelial cells, adult cardiomyocytes, fibroblasts, smooth muscle cells, adult cardiac fibroblasts+5-aza, genetically modified cells, tissue engineered grafts, MyoD scar fibroblasts, pacing cells, embryonic stem cell clones, embryonic stem cells, fetal or neonatal cells, immunologically masked cells, and teratoma derived cells.

Any of the therapeutic agents may be combined to the extent that such combination is biologically compatible.

Although the invention is described with reference to a self-expandable stent, the coating applicator can be used on other medical devices to coat the medical devices during delivery. Non-limiting examples of self-expandable medical devices that may be used according to the present invention include neurocoils, vena cava filters, filters, grafts, and heart valves. It is also possible that the invention could be adapted for use with non self-expandable medical devices which can include stents (balloon expandable or otherwise), catheters, guide wires, balloons, filters (e.g., vena cava filters), stent grafts, vascular grafts, intraluminal paving systems, pacemakers, electrodes, leads, defibrillators, joint and bone implants, spinal implants, access ports, intra-aortic balloon pumps, heart valves, sutures, artificial hearts, neurological stimulators, cochlear implants, retinal implants, and other devices that can be used in connection with therapeutic coatings. Such medical devices are implanted or otherwise used in body structures, cavities, or lumens such as the vasculature, gastrointestinal tract, abdomen, peritoneum, airways, esophagus, trachea, colon, rectum, biliary tract, urinary tract, prostate, brain, spine, lung, liver, heart, skeletal muscle, kidney, bladder, intestines, stomach, pancreas, ovary, uterus, cartilage, eye, bone, joints, and the like.

In the case, for example, of non-self expandable systems, a spring or pressure system could be added to insure that there is still a contact force between the coating applicator and the medical device. Thus, for example, the system may be adapted to coat a balloon-expandable stent crimped on a balloon. In an embodiment similar to that illustrated in FIGS. 2a-2b, the annular ring 20 may be made as a highly elastic tube with a number of longitudinal channels in the wall of the tube, making the inner diameter of the tube to be smaller than the outer diameter of crimped stent on the balloon. The tube may be placed over the stent/balloon assembly by unrolling it over the stent from the proximal side. A needle may then be used to fill the channels of the tube with a suitable therapeutic agent. Upon delivery of the stent in the body, the tube is then unrolled or pulled back over the stent from the distal side, whereby the channels are squeezed. That forces the therapeutic agent to come out whereby it is applied over the stent.

If desired, the medical device may also contain a radio-opacifying agent within its structure to facilitate viewing the medical device during insertion and at any point while the device is implanted. Non-limiting examples of radio-opacifying agents are bismuth subcarbonate, bismuth oxychloride, bismuth trioxide, barium sulfate, tungsten, and mixtures thereof.

The examples described herein are merely illustrative, as numerous other embodiments may be implemented without departing from the spirit and scope of the exemplary embodiments of the present invention. Moreover, while certain features of the invention may be shown on only certain embodiments or configurations, these features may be exchanged, added, and removed from and between the various embodiments or configurations while remaining within the scope of the invention. Likewise, methods described and disclosed may also be performed in various sequences, with some or all of the disclosed steps being performed in a different order than described while still remaining within the spirit and scope of the present invention.

We claim:

1. A system for deploying a self-expandable stent with a coating, comprising:
a self-expandable stent;
a tubular sheath having an opening at a distal end of the sheath, said self-expandable stent loaded inside of said tubular sheath; and
at least one coating applicator mounted at the distal end of the sheath and adapted to apply coating material to the self-expandable stent as it exits the sheath through the opening at the distal end of the sheath.

2. The system of claim 1, wherein the coating applicator comprises a hollow ring adapted to contain coating material, wherein the hollow ring has an inner surface with holes in the inner surface for allowing coating material to pass from the applicator to the stent.

3. The system of claim 2, wherein the inner surface of the applicator is deformable, and wherein the stent exerts a pressure on the deformable inner surface during deployment, thereby causing coating material to be expelled through the holes onto the stent.

4. The system of claim 2, wherein the holes are evenly distributed.

5. The system of claim 2, wherein the holes are not evenly distributed.

6. The system of claim 2, wherein the holes are of uniform size.

7. The system of claim 2, wherein the holes are not of uniform size.

8. The system of claim 2, wherein the hollow ring has an outer surface, and wherein the outer surface is hard.

9. The system of claim 1, wherein the coating applicator comprises a housing containing a plurality of spherical balls, wherein said housing is adapted to contain coating material and allows rotation of the spherical balls to apply the coating material to the stent as it exits the sheath.

10. The system of claim 9, wherein the coating applicator further comprises a first ring of spherical balls and a second ring of spherical balls.

11. The system of claim 10, wherein at least some of the spherical balls in the second ring of spherical balls are staggered with respect to at least some of the spherical balls in the first ring of spherical balls.

12. The system of claim 1, wherein the coating applicator comprises a gel, and wherein when the stent exits the sheath, the stent exerts a force on the gel causing gel to be sheared off and coated onto the outer surface of the stent.

13. The system of claim 1, wherein the coating material comprises a therapeutic agent.

14. The system of claim 1, wherein the at least one coating applicator comprises a plurality of coating applicators for forming a multi-layer coating.

15. The system of claim 14, wherein the plurality of coating applicators comprises:
a first coating applicator adapted to apply a first coating material to the stent; and
a second coating applicator adapted to apply a second coating material to the stent;
wherein the first coating applicator is proximal to the second coating applicator.

16. The system of claim 15, wherein the first coating material comprises a slow release drug and the second coating material comprises an immediate release drug.

17. The system of claim 1, wherein the coating applicator comprises a reservoir.

18. A method of deploying a self-expandable stent with a coating, comprising the steps of:
inserting a self-expandable stent into a tubular sheath, said sheath comprising an opening at a distal end of the sheath and at least one coating applicator mounted at the distal end of the sheath;
inserting the sheath with the stent inside the sheath into a lumen of a patient;
deploying said stent from the opening at the distal end of the sheath, thereby applying coating material to the stent from the coating applicator as the stent passes by the coating applicator; and
allowing the stent to self-expand as it exits from the sheath.

19. The method of claim 18, wherein the step of applying coating material to the stent from the coating applicator as the stent passes by the coating applicator comprises applying a first coating material and a second coating material to the stent.

20. A method of deploying a self-expandable medical device with a coating, comprising the steps of:
inserting a self-expandable medical device into a tubular sheath, said sheath comprising an opening at a distal end of the sheath and at least one coating applicator mounted at the distal end of the sheath;
inserting the sheath with the medical device inside the sheath into a lumen of a patient;
deploying said medical device from the opening at the distal end of the sheath, thereby applying coating material to the medical device from the coating applicator as the medical device passes by the coating applicator; and
allowing the medical device to self-expand as it exits from the sheath.
